(19) United States
(12) Patent Application Publication
(10) Pub. No.: US 2007/0164050 A1
(43) Pub. Date: Jul. 19, 2007

(54) Personal Antimicrobial Dispenser

(76) Inventor: Timothy Delaine Knight, Chesapeake, VA (US)

Correspondence Address: TIMOTHY DELAINE KNIGHT, 1001 FALLHAVEN CT., CHESAPEAKE, VA, 23320, US

(21) Appl. No.: 11/624,350

(22) Filed: Jan. 18, 2007

Related U.S. Application Data
(60) Provisional application No. 60/760,183, filed on Jan. 19, 2006.

(51) Int. Cl. B67D 5/64

(52) U.S. Cl. 222/175

(57) ABSTRACT

A personal antimicrobial dispenser that can be easily carried on a persons belt, waist line, garment or baggage by the use of a clipping device that is attached to the dispensing bottle. The dispenser bottle is refillable and can be filled with store bought antimicrobial solutions or various other liquid to gel like solutions such as mosquito repellants. When the dispenser is mounted on a belt or waist line it is easily used by pulling a lever horizontally outward from the body and catching the solution dispensed from the bottle in the same hand that activates the lever. The dispenser has a face plate that can be attached directly to the dispensing bottle allowing the bottle to be hidden or disguised for aesthetic or functional reasons. The dispenser faceplate can be modified to act as a cell phone carrier.

### CROSS-REFERENCE TO RELATED APPLICATIONS

This application claims the benefit of PPA Ser. No. 60/760,183, filed Jan. 1-19, 2006 by the present inventor, which is incorporated by reference.

### FEDERALLY SPONSERED RESEARCH

Not Applicable.

### SEQUENCE LISTING OR PROGRAM

Not Applicable.

### BACKGROUND

1. Field of Invention

The present invention relates to the need of an individual to be able to dispense an antimicrobial solution on to their hand or hands in a quick and efficient movement while only needing to use one hand in the process. Viruses, germs and bacteria can be found on all surfaces that are touched several times a day by individuals as they are working, playing or traveling. Casual contact with these contaminated surfaces allows for the rapid spread of dangerous viruses, bacteria and germs.

2. Prior Art

Most current antimicrobial dispensers are simply copies of pump action washing detergent containers in smaller sizes. These dispensers have several disadvantages:
(a) The dispenser must be carried inside a bag or other type of carrying case
(b) Since the dispensers are inside bags and other cases the ability to use it is greatly hampered by the user having to fumble through the bag looking for the dispenser.
(c) The time and complications of physically getting to the dispenser means that the antimicrobial solution is much less likely to be used in a continuous pattern that is essential to keeping ones hands clean of bacteria, viruses and germs.

There are a few devices that are body mounted but the dispensers have design flaws that will limit the user's ability to actually use the device or prevent the manufacturer from making the dispenser at a profitable price. U.S. Pat. No. 5,927,548 is designed to attach to a waist line but implements the use of a removable cartridge and a complex bladder type system for dispensing the solution on to the users hand. U.S. Pat. No 6,283,334 is also designed to be waist mounted but it requires the use of two hands for dispensing the solution by squeezing the container with one hand and catching the fluid in the other. The device also requires that the container be inverted and mounted to the holster before use. There are various other dispensers that are worn like a wrist watch but are cumbersome to wear and use.

The aforementioned patents also were designed with working professionals in mind while my dispenser is designed for the casual user with visual aesthetics taken in to account by adding a faceplate for attractiveness. My dispenser also will not accidentally dispense a solution when leaned on because the action to dispense the solution is by pulling a lever from the body side outward.

### SUMMARY

In accordance with one embodiment a solution dispenser that can be attached to a persons belt, garment, bag or other carried article of clothing. The solution dispenser can be used with one hand and allows for attachment of designer face plates for attractiveness in appearance.

### DRAWINGS—FIGURES

FIG. 1 is a perspective front view of the solution dispenser without the face plate interconnected.

FIG. 2 is a perspective back view of the solution dispenser without the face plate interconnected.

FIG. 3 is a three dimensional view of a face plate.

FIG. 4 is a two dimensional side view of the dispenser showing how the face plate interconnects with the solution dispenser.

FIG. 5 is a two dimensional side view of a face plate modified to also be a cell phone holder.

### DRAWINGS—REFERENCE NUMERALS

10 Cap on container
20 Solution container
30 Dispensing lever
40 Dispensing tip
50 Face plate attachment devices
70 Mounting Clip
90 Face plate
100 Generic pump
110 Face plate for holding a cell phone

### DETAILED DESCRIPTION OF DRAWINGS

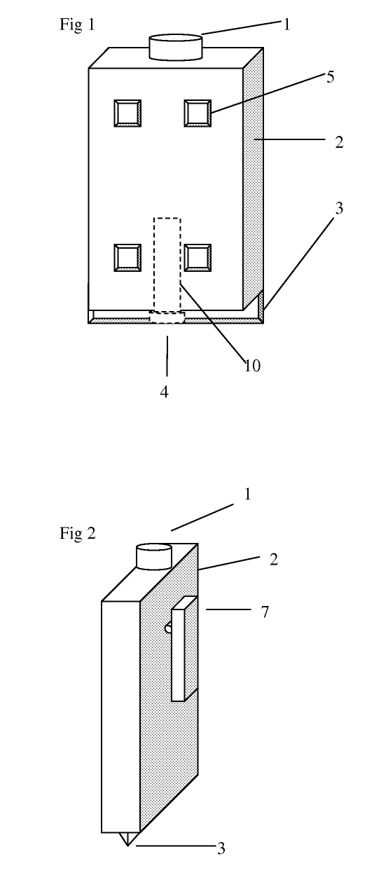

FIG. 1 is a perspective view of the front side of the dispenser apparatus. The solution container 20 is small enough to easily be worn or carried around by attaching it to a persons clothing or carry bag. The cap 10 of the solution container 20 is either a screw on cap or press in cap, which allows for refilling the container 20 with typical store purchased sanitizers. The dispensing lever 30 is used to release the liquid from the solution container 20 on to the user hand. The user pulls the lever 30 horizontally away from the user body to dispense the solution that is in the solution container 20. This design ensures that the solution dispenser will not accidentally release any solution when the solution dispenser is leaned on by the user.

The dispensing tip 40 and generic pump 100 is located at the bottom of the solution container 20. When the dispensing lever 30 is pulled outward horizontally from the body the solution released from inside the solution container 20 will land on the user hand. This design allows for one hand usage. The same hand that pulls the dispensing lever 30 is also catching the solution as it is released from the solution container 20. The pump 100 and tip 40 used will be commercially existing devices that will be chosen based on cost, quality, design and the ability to fit the pump 100 inside the solution container 20 or attach it to the bottom of the solution container. No specific drawings of how the dispensing lever 30 is mounted to the solution container 20 or the exact appearance of the dispensing lever 30 are shown because this may vary slightly depending on the pump 100 installed inside the solution container 20.

The face plate attachment devices 50 hold the decorative face plate 90 to the solution container 20. The face plate attachment devices 50 can be made of various substances like Velcro, plastic clips, snap on clips, screws or even simple knobs that allow the face plate 90 to interconnect securely from or to the solution container 20. This allows for the solution container 20 to be disguised or hidden while being worn or carried on a bag in public places. The face plate 90 can also be used to encourage younger children to carry the solution dispenser on them by the use of cartoon or popular characters in the design.

FIG. 2 is a perspective back side view of the solution dispenser. This view shows that the solution dispenser is thin enough that it is non obtrusive to the user when it is worn on a belt or carry baggage. A standard mounting clip 70 such as an alligator clip or spring loaded clip is attached to the solution container 20 using glue, screws or other means that will ensure that the solution container is attached securely to the clip. The style or type of mounting clip 70 will be chosen based on price, installation costs and aesthetic appeal. The solution container cap 10 is located at the top of the solution container and is used to refill the solution container when the solution runs out. This allows the solution container 20 to be used multiple times and also allows for the solution dispenser to be used as more than just a sanitizer. For instance the solution container 20 could be filled with a mosquito repellant or any other solution that will work properly with the installed pump.

FIG. 3 is a perspective view of the face plate 90. The face plate attachment devices 50 securely hold the face plate 90 to the solution container 20. The face plate 90 can be made of plastic, metal or any other rigid type substance that can be formed, molded or shaped in to appealing shapes and colors. The purpose of the face plate 90 is to hide, disguise or make the solution dispenser more aesthetically pleasing, fun and functional for users to carry on their body or baggage.

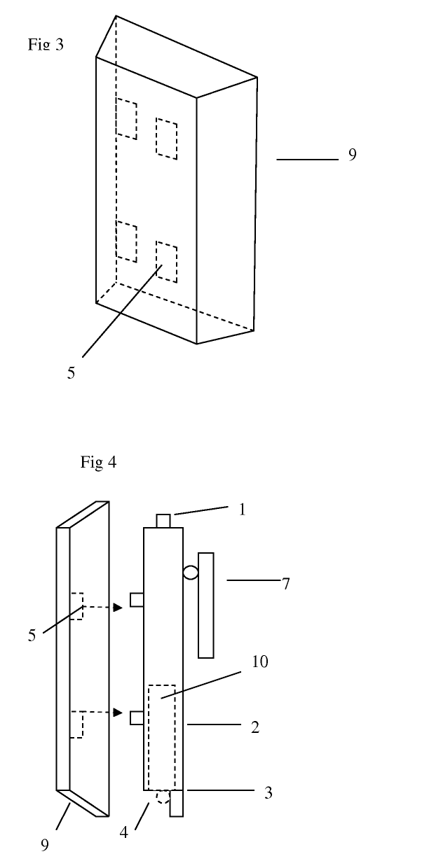

FIG. 4 is a two dimensional side view of the solution container 20 and face plate 90. This view shows the orientation of the face plate 90 to the solution 20 container when they are interconnected together.

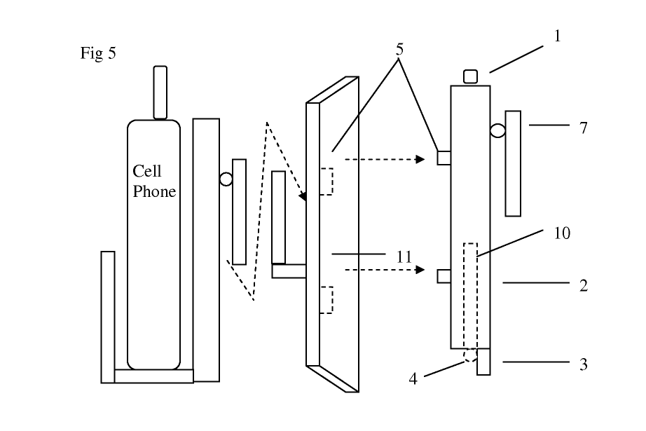

FIG. 5 is a two dimensional view of face plate 110 that has been designed to hold a cell phone. This allows for the solution dispenser and cell phone to be carried with the same clip.

I claim:

1. A solution container comprising a refillable container with a means of dispensing a solution from within said container with the use of only one hand and said container being mountable on a human for ease of carry and an optional face plate covering said container for design, use and aesthetic purposes.

2. The opening at top of said container of claim 1 has a screw on, press on cap or other means of securing a cap for temporarily sealing said container and allowing said container to be refilled multiple times.

3. The bottom of said container of claim 1 has a dispensing device attached that dispenses solution from within said container when a lever is activated by the user wherein said lever communicates with said dispensing device to release a predetermined amount of solution.

4. The dispensing device of claim 3 is attached to the bottom or mounted inside of said container by a means that seals said container and allows for a solution to be dispensed in a predetermined amount when said lever of claim 3 is communicated with by the user.

5. The lever of claim 3 is pulled with one hand outward and communicates with said dispensing device of claim 3 and releases a predetermined amount of solution on to the same hand that pulls said lever.

6. The solution container of claim 1 is mountable on a human pant waist line, belt, pocket, baggage or communication device holder by the use of a mounting clip that is permanently or temporarily attached to the inward side said solution container.

7. The face plate of claim 1 is attached to said solution containers outward side by a means that allows for securely holding said face plate to said solution container.

8. The face plate of claim 1 wherein said design is detachable or permanently attached to said solution container of claim 7 and of which the face plates shape, color, function or size is variable.

9. The face plate of claim 1 wherein said design may be attached to a cell phone or other communication device.
